(19) United States
(12) Patent Application Publication
(10) Pub. No.: US 2024/0325124 A1
(43) Pub. Date: Oct. 3, 2024

(54) METHOD FOR PROVIDING ANNOTATED SYNTHETIC IMAGE DATA FOR TRAINING MACHINE LEARNING MODELS

(71) Applicant: Milan Madhavji, Mississauga (CA)

(72) Inventor: Milan Madhavji, Mississauga (CA)

(21) Appl. No.: 18/616,409

(22) Filed: Mar. 26, 2024

Related U.S. Application Data
(60) Provisional application No. 63/493,797, filed on Apr. 3, 2023.

(51) Int. Cl. A61C 9/00; G06T 17/00; G06V 20/70; G16H 50/50

(52) U.S. Cl. CPC A61C 9/0053; G06T 17/00; G06V 20/70; G16H 50/50

(57) ABSTRACT

A method for training a machine learning model includes the steps of generating a three-dimensional model of at least one anatomical structure, labeling data of the three-dimensional model to identify modeled substructures of the at least one anatomical structure, acquiring a plurality of two-dimensional images from the three-dimensional model, and inputting the plurality of two-dimensional images as training data to train a machine learning model to identify substructures of the at least one anatomical structure in an image.

### CROSS-REFERENCE TO RELATED APPLICATIONS

This application claims the benefit of U.S. Provisional Application No. 63/493,797 filed Apr. 3, 2023, the contents of which are incorporated herein by reference.

### TECHNICAL FIELD

The present invention relates to a method for providing annotated synthetic image data for training machine learning models. More specifically, the present invention relates to a method for providing annotated synthetic image data using a three-dimensional model of dentoalveolar structures and conditions for training of machine learning models in the field of image processing for dental radiology.

### BACKGROUND

Machine learning (ML) is rapidly becoming more integral to performing day-to-day tasks in the workplace. Its growing number of applications is changing how businesses operate, make decisions, and improve efficiency. Machine learning relies on algorithms that can learn from and make predictions or decisions based on data. The process of training a machine learning algorithm involves feeding these algorithms large sets of training data so that they can automatically learn and improve from experience without being explicitly programmed for each task.

Training data refers to the dataset used to teach or instruct a machine learning model so that it can understand, learn, and make predictions or decisions based on new, unseen data. Training data is the foundational information that provides the example inputs and corresponding outputs for the machine learning algorithm to process. This data is used during the training phase, where the model iteratively adjusts its parameters to minimize the difference between its predictions and the actual outcomes provided by the training data. Training data is crucial for the performance of a machine learning model, as the quality, quantity, and diversity of the data directly influence how well the model can generalize its learning to perform accurately on data it has never seen before.

In the domain of machine learning models for image processing, training data for such models typically comprises a large array of images that have been labeled or annotated to help the model understand and interpret visual information. The complexity of image processing tasks that can be conducted by a trained model may range from simple object recognition to more intricate challenges such as recognition of aberrant conditions and autonomous detection of disease states in humans.

The quality of training data is very important. For machine learning models to function effectively in real-world scenarios, they must be trained on diverse and representative datasets. This diversity includes variations in, to name a few variables, pixel intensity, angles, backgrounds, and object appearances in images, ensuring that the model can generalize well across different settings and conditions. The process of collecting, labeling, and curating such datasets is both time-consuming and labor-intensive, often involving manual annotation by humans to provide accurate labels for the objects or features present in each image.

However, the process of assembling such training datasets has many challenges. Among the most tedious aspects of training machine learning models for the recognition of dentoalveolar structures and conditions is the annotation of real scan images for use in training the machine learning models. In dental radiology, scan images of the dentoalveolar structure of a patient are annotated by radiologists with descriptive information relating to characteristics shown in the scan images. By way of example, image data indicative of lesions, caries, pathology, physical abnormalities or other clinically relevant information may be annotated by the radiologist in order to convey descriptive information to other individuals who may attend to the patient. Often, the annotations may be imprecise. Such imprecision may be due to the nature of human error or human bias in making the annotations. Bias and error can adversely affect the performance of the trained model by skewing its predictions made on live data. When the data annotation process is influenced by bias or error, the resulting dataset may not accurately represent the diversity of real-world scenarios, leading to a trained model that performs sub-optimally in certain situations.

Another challenge is that the annotated training data set may not cover an adequate gamut of possibilities. The body of annotated training data related to certain conditions is dependent upon the frequency of patients with those conditions. Image data for rare conditions is likewise rare. As a result, the quality of the predictive output of a machine learning model trained on annotated image information is limited by the precision and availability of annotation and labeling of the training data.

In the field of dental radiology, images of patient dentoalveolar and intraoral structures are often taken by imaging processes such as cone-beam computed tomography (CBCT). Existing cone-beam computed tomography (CBCT) scans are not ideal for use as training data because the issues and limitations associated with manual annotations are still present. Moreover, the resolution of CBCT scans is too low to produce images of adequate quality for the creation of two-dimensional (2D) images that would mimic intraoral and extraoral plain film scans. Use of high-quality, clear images as training data ensures that the model can accurately identify and understand the features and patterns present in the data, which is essential for tasks such as object detection and image classification. Clear training data helps in reducing noise and ambiguity, enabling the model to make more precise predictions. If the training images are blurry, distorted, or otherwise unclear, the model might be delayed in learning or fail to learn the relevant features, leading to poor performance on real-world data.

A means of providing accurately annotated training data for training a machine learning model for image processing, which is both precise and highly available, is therefore desirable.

### SUMMARY

The present invention relates to a method for providing annotated synthetic image data for training machine learning models. More specifically, the present invention relates to a method for providing annotated synthetic image data using a three-dimensional model of dentoalveolar structures and conditions for training of machine learning models in the field of image processing for dental radiology.

From the 3D model, all types of two-dimensional (2D) and three-dimensional (3D) dental x-ray scan images may be mimicked perfectly. More specifically, synthetic 2D and synthetic 3D images representing the patient data from any perspective may be generated using the 3D model. The synthetically generated x-ray scan images produced with this 3D model are perfectly and automatically annotated. Moreover, the synthetically generated x-ray scan images produced using the 3D model are capable of being tuned to produce images that mimic the various types of dental x-rays produced for each type of modality such as, for example, plain films, PSP sensor, CMOS sensor, image intensifier, or other modalities. Therefore, the 3D model may be mined to quickly produce a large quantity of high-quality data useful for enabling quick and efficient training of high-quality predictive machine learning models for identification of various dental states and conditions of interest.

The 3D model may include or can be modified to include representations of associated conditions and states of the patient, such as various diseases at various stages, morphologic variants of anatomy, dental treatments and normal growth. With synthetic 3D modeling of a sufficient quantity of patients, the body of image data available may represent a wide variety of conditions or injuries, both commonplace and rare, oral structures in various stages of development and healing, and including a wide variety of anatomical variability.

A synthetic dentoalveolar model is provided which can be adjusted to mimic the entire spectrum of dental appearances in real images. This synthetic model would be useful for emulating at least one of normal appearances, normal growth, simulations of abnormal situations, and abnormal growth. Examples of abnormal situations and abnormal growth which may be emulated using the 3D model may include a tooth bud that is malpositioned or incorrectly oriented. Such a tooth bud would grow into an ectopic position and would affect the adjacent teeth. As another example, hindered growth of a specific structure or bone would mimic a real dental condition. Other examples include microdont and hemifacial microsomia. Moreover, the 3D model may be used to model disease processes such that the nascent disease can be simulated at any location. The subsequent timeline of disease progression can also be emulated by the model. This would simulate a realistic outcome, with realistic effects on the surrounding structures.

In one aspect, there is provided a method for acquiring training data for training a machine learning model. The method includes the steps of generating a three-dimensional model of at least one anatomical structure, labeling data of the three-dimensional model to identify modeled substructures of the at least one anatomical structure, and acquiring a plurality of two-dimensional images from the three-dimensional model to be input as training data to train a machine learning model useful for identification of substructures of the at least one anatomical structure in an image.

In one aspect, the image is a radiographic image. The radiographic image may be a radiographic image of at least one dentoalveolar structure of a patient.

The step of acquiring a plurality of two-dimensional images may further include the step of taking a plurality of cross-section images from portions of the three-dimensional model.

The step of generating a three-dimensional model of at least one anatomical structure may further include the steps of acquiring a volumetric scan of the at least one anatomical structure, and, constructing a three-dimensional model of the at least one anatomical structure. In one aspect, the step of constructing the three-dimensional model may further include the step of manually constructing at least a portion of the three-dimensional model. In another aspect, the step of constructing the three-dimensional model may further include the step of procedurally generating at least a portion of the three-dimensional model. In yet another aspect, the step of constructing the three-dimensional model may further include the step of independently modeling each modeled substructure of the three-dimensional model and then assembling the modeled substructures together to form the three-dimensional model.

The step of acquiring the volumetric scan may further include the step of scanning at least one anatomical structure of a patient to acquire the volumetric scan.

In one aspect, after the generating step, the method further includes the step of applying a greyscale gradient to voxels of the three-dimensional model. The method may further include the step of inverting the greyscale gradient.

After the generating step, the method may further include the step of applying at least one color to voxels of the modeled substructures of the three-dimensional model. In one aspect, each of the modeled substructures is independently colored.

After the generating step, the method may further include the step of selectively rendering at least partially transparent voxels of the three-dimensional model.

In one aspect, the modeled substructures include at least one modeled tooth structure including at least one of modeled enamel, modeled dentin, modeled pulp cavity, modeled pulp, modeled alveolar ligament space, and modeled root cavity. The modeled substructures may include at least one modeled bone structure including at least one of modeled trabeculae, modeled lamina dura and modeled cortical boundaries. In yet another aspect, substructures of the three-dimensional model are at least one of removeable and isolatable from the three-dimensional model.

In one aspect, the method may further include the step of outlining at least one modeled substructure of the three-dimensional model. In another aspect, the method further includes the step of highlighting at least one modeled substructure of the three-dimensional model.

In another aspect, there is provided a method for training a machine learning model including the steps of generating a three-dimensional model of at least one anatomical structure, labeling data of the three-dimensional model to identify modeled substructures of the at least one anatomical structure, acquiring a plurality of two-dimensional images from the three-dimensional model, and inputting the plurality of two-dimensional images as training data to train a machine learning model to identify substructures of the at least one anatomical structure in an image.

In one aspect, the image is a radiographic image. The radiographic image may be a radiographic image of at least one dentoalveolar structure of a patient.

The step of acquiring a plurality of two-dimensional images may further include the step of taking a plurality of cross-section images from portions of the three-dimensional model.

The step of generating a three-dimensional model of at least one anatomical structure may further include the steps of acquiring a volumetric scan of the at least one anatomical structure, and, constructing a three-dimensional model of the at least one anatomical structure. In one aspect, the step of constructing the three-dimensional model may further include the step of manually constructing at least a portion of the three-dimensional model. In another aspect, the step of constructing the three-dimensional model may further include the step of procedurally generating at least a portion of the three-dimensional model. In yet another aspect, the step of constructing the three-dimensional model may further include the step of independently modeling each modeled substructure of the three-dimensional model and then assembling the modeled substructures together to form the three-dimensional model.

The step of acquiring the volumetric scan may further include the step of scanning at least one anatomical structure of a patient to acquire the volumetric scan.

In one aspect, after the generating step, the method further includes the step of applying a greyscale gradient to voxels of the three-dimensional model. The method may further include the step of inverting the greyscale gradient.

After the generating step, the method may further include the step of applying at least one color to voxels of the modeled substructures of the three-dimensional model. In one aspect, each of the modeled substructures is independently colored.

After the generating step, the method may further include the step of selectively rendering at least partially transparent voxels of the three-dimensional model.

In one aspect, the modeled substructures include at least one modeled tooth structure including at least one of modeled enamel, modeled dentin, modeled pulp cavity, modeled pulp, modeled alveolar ligament space, and modeled root cavity. The modeled substructures may include at least one modeled bone structure including at least one of modeled trabeculae, modeled lamina dura and modeled cortical boundaries. In yet another aspect, substructures of the three-dimensional model are at least one of removeable and isolatable from the three-dimensional model.

In one aspect, the method may further include the step of outlining at least one modeled substructure of the three-dimensional model. In another aspect, the method further includes the step of highlighting at least one modeled substructure of the three-dimensional model.

### BRIEF DESCRIPTION OF THE DRAWINGS

FIG. 1 is a flowchart illustrating a method for training a machine learning model;

FIG. 2 is an illustration of a tooth showing substructures thereof;

FIG. 3 is a radiographic image of a tooth with substructures labeled;

FIG. 4 is a side-by-side comparison of a cone-beam computed tomography (CBCT) scan with an original periapical radiograph of the same structure;

FIG. 5 is a representation of teeth generated from a synthetic 3D model;

FIG. 6 is the three-dimensional model shown in FIG. 5 with inverted contrast;

FIG. 7 is a magnified portion of a three-dimensional model of a dental structure;

FIG. 8 is a three-dimensional model of a dentoalveolar structure;

FIG. 9 is the three-dimensional model shown in FIG. 8 with inverted contrast;

FIG. 10 is a magnified portion of the three-dimensional model shown in FIG. 9;

FIG. 11 is a bitewing radiographic image of a dentoalveolar structure;

FIG. 12 is a top-down perspective view of a three-dimensional model of dentition;

FIG. 13 is the perspective view of FIG. 12 with inverted contrast;

FIG. 14 is a three-dimensional model of dentition with an abnormality according to one aspect;

FIG. 15 is a three-dimensional model of dentition with an abnormality according to one aspect;

FIG. 16 is a three-dimensional model of dentition with an abnormality according to one aspect;

FIG. 17 is a three-dimensional model of dentition with an abnormality according to one aspect;

FIG. 18 is a three-dimensional model of dentition with an abnormality according to one aspect;

FIG. 19 is a 3D model of a mandible according to one aspect of the present invention;

FIG. 20 is the 3D model of the mandible shown in FIG. 19 with inverted contrast;

FIG. 21 is a portion of the 3D model shown in FIG. 19 at higher magnification;

FIG. 22 is the portion of the 3D model of FIG. 21 with inverted contrast;

FIG. 23 is a 3D model according to one aspect of the present invention with annotated data;

FIG. 24 is a portion of the 3D model of FIG. 23 with pulp highlighted;

FIG. 25 is a portion of the labeled 3D model of FIG. 23 with enamel highlighted;

FIG. 26 is a flowchart illustrating a method for acquiring training data for training a machine learning model, according to one aspect; and,

FIG. 27 is a flowchart illustrating a method for training a machine learning model, according to one aspect.

### DETAILED DESCRIPTION

The present invention relates to a method for providing annotated synthetic image data for training machine learning models. More specifically, the present invention relates to a method for providing annotated synthetic image data using a three-dimensional model of dentoalveolar structures and conditions for training of machine learning models in the field of image processing for dental radiology.

These and other features and advantages are described in, or are apparent from, the following detailed description of various exemplary embodiments.

It will be understood that, although the terms “first”, “second”, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of exemplary embodiments.

In the drawing figures, the dimensions of layers and regions may be exaggerated for clarity of illustration. Like reference numerals refer to like elements throughout. The same reference numbers indicate the same components throughout the specification.

Spatially relative terms, such as “beneath,” “below,” “lower,” “above,” “upper” and the like, may be used herein for ease of description to describe one element or feature's relationship to another element(s) or feature(s) as illustrated in the figures. For example, elements described as “below” or “beneath” other elements or features would then be oriented “above” the other elements or features. Thus, the exemplary term “below” can encompass both an orientation of above and below. The elements may be otherwise oriented (rotated 90 degrees or at other orientations) and the spatially relative descriptors used herein interpreted accordingly.

The terminology used herein is for the purpose of describing particular aspects only and is not intended to be limiting of exemplary embodiments. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Exemplary embodiments are described herein with reference to cross-sectional illustrations that are schematic illustrations of idealized embodiments (and intermediate elements) of exemplary embodiments. As such, variations from the shapes of the illustrations as a result, for example, of different scanning modalities and/or modeling, are to be expected. Thus, exemplary embodiments should not be construed as limited to the particular shapes of regions illustrated herein but are to include deviations in shapes that result, for example, from data collection and representation. For example, an implanted region illustrated as a rectangle will typically have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the figures are schematic in nature and their shapes are not intended to illustrate the actual shape of a region of an element and are not intended to limit the scope of exemplary embodiments.

Unless otherwise defined, all terms (including technical and scientific terms) used herein have the same meaning as commonly understood by one of ordinary skill in the art to which exemplary embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein. As used herein, expressions such as “at least one of,” when preceding a list of elements, modify the entire list of elements and do not modify the individual elements of the list.

Reference will now be made in detail to aspects, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain exemplary embodiments of the present description.

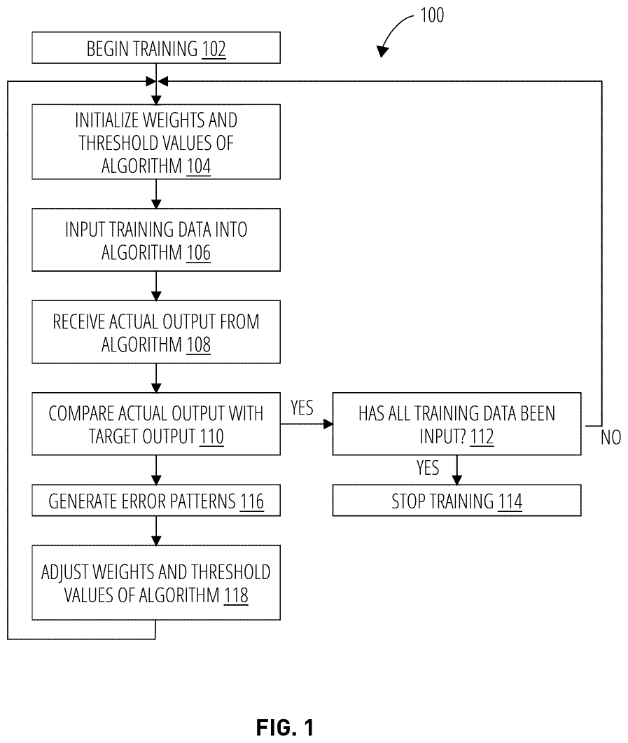

FIG. 1 illustrates a flow diagram of a process 100 for training a machine learning model for analyzing radiographic images using a machine learning algorithm, in accordance with one aspect. One of ordinary skill in the art would appreciate that the processing is conducted using one or more computers having a plurality of processors and system architecture for executing the machine learning analytical processes described herein, embodied in at least one software program, a plurality of storage devices or a data management system for storing the requisite data, library information, and other information for conducting these analyses, and at least one output device, such as one or more other computing devices, servers or data management systems, networks, “cloud” systems, monitors or other computing devices and peripherals. It should also be understood that the software including the machine learning model may be housed on a computer system or data management system at a remote location. Data produced by or retrieved from the system or components thereof may be sent via a suitable network connection to the remote computer system or data management system for processing. The output of the model may then be sent back to the location of the system for review by a qualified person or automated system.

At block 102, training is initiated. At block 104, the weights and thresholds of the machine learning algorithm are initialized. The weights and thresholds may be initialized with, for example, random or arbitrary numbers that are within reason to a person skilled in the art. Weights and thresholds are fundamental concepts in training machine learning algorithms, particularly in the context of models that simulate a decision process.

“Threshold” refers to a cutoff value that determines the decision boundary for the output of a model. In the context of a binary classification problem, for example, the model might output a probability that a given input belongs to a particular class. The threshold is then used to decide whether this probability is sufficient to classify the input as belonging to that class or not. If the output of the model exceeds the threshold, the input might be classified as positive; otherwise, it is classified as negative. Thresholds are important for tailoring the model sensitivity of a model to its specific application allowing balance of the model between concerns such as precision and recall based on the problem at hand.

“Weight” refers to parameters, typically numerical parameters, that are assigned to inputs in the model. They signify the importance or influence of each input on the model predictions of the model. During the training process, the model adjusts these weights based on the input data it receives, seeking to minimize the difference between its predictions and the actual target outcomes. This process is aimed at capturing the underlying patterns and relationships within the data. In short, weights determine how much each input feature contributes to the model's output.

During training, the model iteratively adjusts its weights to minimize the error between its predictions and the actual outcomes. This process may involve a loss function, which quantifies the error of the model, and an optimization algorithm, which dictates how the model should adjust its weights to reduce this error. The threshold might be adjusted post-training, based on performance metrics on a validation set, to fine-tune the performance of the model according to specific requirements.

In short, weights and thresholds are important for defining and adjusting the decision-making process of the machine learning model, enabling it to learn from data and make accurate predictions.

At block 106, a set of training data is input to the machine learning algorithm. The training data may include images taken from the synthetic three-dimensional model, as described hereinafter. Preferably, the training data is suitably labeled or annotated. In the context of machine learning, the term “annotation” refers to the process of labeling or tagging data with tags, labels, or classifications that render the data more meaningful or insightful for machine learning algorithms. This process entails assigning metadata to various data types, such as substructures in images of anatomical structures, thereby providing a structured framework for machine learning algorithms to understand and interpret the underlying patterns within the image data. Through the provision of annotated data, machine learning models are able to learn efficiently and perform accurately.

The algorithm makes use of the assigned weights and thresholds to generate at least one output based on a suitable resolving function. An actual output of a classified image is received at block 108. The output may, for example, be in the form of differentiable signals such as numerals between, 0 and 1, in the form of positive or negative states implied by an output numeral of greater than or less than 0 respectively, or any other suitable indication as evident to a person of ordinary skill in the art.

At block 108, the actual output is compared with the desired or target output, defined by the person training the model with knowledge of whether the input training data is or is not representative of a specific image classification outcome. If the actual output is commensurate with the desired or target output or, if the difference between the target and actual output falls below a predefined acceptable level, a check is made at block 112 to determine whether the neural network has been trained on the entire set of training data. If not, then the weights and thresholds of the algorithm are initialized at block 104 and the next set of training data is introduced to the neural network at block 106 and the foregoing steps outlined in block 102 and block 106 to block 112 are repeated. The training process continues until the machine learning model has been trained on the entire set of training data.

If the comparison at block 110 suggests that the actual output is not in agreement with the desired or targeted output, the ensuing additional steps are performed. At block 116, the difference between the actual output and the target output is used to generate an error pattern in accordance with a suitable back-propagation rule or any other suitable error estimation rule known to a person of ordinary skill in the art. The error pattern is used to adjust, at block 118, weights and thresholds in the machine learning algorithm such that the error pattern would be reduced at the next instance the training process is performed, if the same set of training data were presented as the input data. Once the weights and threshold values of the algorithm have been adjusted, the next set of training data is introduced to the neural network to iterate through the learning cycle again. The algorithm is therefore trained by presenting each set of training data in turn at the inputs and propagating forwards and backwards, followed by the next input data, and repeating this cycle a sufficient number of times such that the weights and threshold values are iteratively adjusted to establish a set of weights and threshold values which may be relied upon to produce a pattern of actual output that is in agreement with the target output for the presented input data. Once the desired set of weights and threshold values is established, preferably when all training data has been input to the algorithm, then the learning process may be terminated, as shown at block 114. The product of the training is a trained machine learning model capable of receiving radiographic image inputs and classifying regions of the image as certain substructures. The learned information of the algorithm is contained in the values of the set of weights and thresholds.

Once the machine learning model has been trained using the training data, then classification of image data may be performed using live input data. Live input data may be provided in the manner described above, for example by way of real radiographic images taken from a patient, for example.

It should be further understood that using a given feature set, the same degree of predictive accuracy can be achieved by many types of known trainable classifiers or neural networks. The training operation can be performed on one machine and the results can be replicated in additional machines. For example, training of a machine learning algorithm results in a set of weight values defining the association between input and output. This set can be recorded and incorporated in other, similar machine learning algorithms.

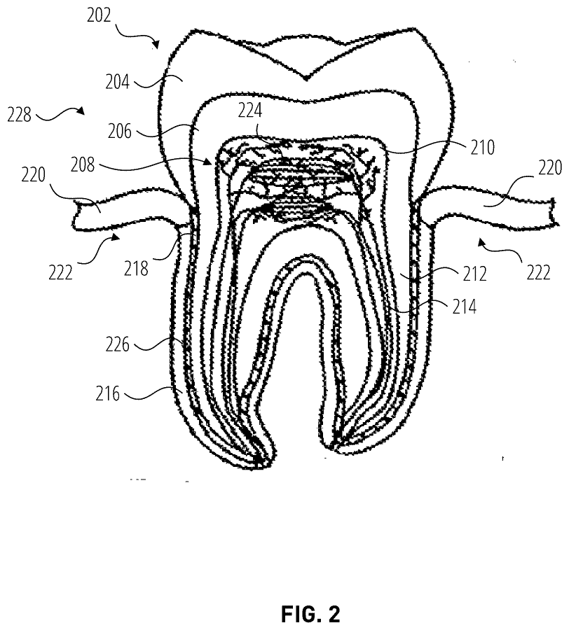

In FIG. 2, there is shown an illustration of the components of a tooth 202 and surrounding anatomical structures. Tooth 202 includes substructures such as the enamel 204, dentin 206, pulp cavity 208 having pulp 210 therein, root 212 having root canal 214 traversing therethrough, periodontal ligament space 216, surrounding gingiva 220, and the alveolar bone 222. Each substructure plays a pivotal role in dental health and functionality. Although only a single tooth 202 is shown in FIG. 2, it should be understood that each tooth 202 of the human mouth includes the same or similar substructures.

Enamel 204 is the outermost layer of tooth 202 and is characterized by its highly mineralized composition. Enamel 204 is a very dense structure which provides a protective layer encapsulating dentin 206 which is a less mineralized, porous tissue that supports the enamel 204 and conveys sensory signals to the pulp cavity 208. The pulp cavity 208 resides at the core of the tooth 202 and houses the vital components of the tooth 202 including nerves and blood vessels 224. The pulp cavity 208 extends into the root canal 214, a narrow channel that traverses the root 212, facilitating the flow of nutrients and signals to and from the pulp 210.

Surrounding the root 212 is the periodontal ligament space 216, which contains the periodontal ligament 218, a connective tissue that anchors the tooth 202 to the alveolar bone 222 surrounding the root 212, cushioning tooth 202 against mechanical stresses and permitting slight mobility. The cementum 226 is an outer layer of the root which provides a surface for the periodontal ligament 218 to connect with the root 212. The gingiva 220, or gum tissue, envelops the alveolar bone 222 and part the root 212 of the tooth 202, providing a protective barrier. The alveolar bone 222 is part of the jawbone (not shown) and embeds tooth 202 therein, providing structural support thereto.

Together, the enamel 204, dentin 206 and at least a portion of the pulp cavity 208 make up the crown portion 228 of the tooth 202 whereas the root canal 214, periodontal ligament space 216 and alveolar bone 222, and, in some instances, a portion of the pulp cavity 208 make up the root 212 of the tooth 202. Between crown portion 228 and root 212 lies the cementoenamel junction 230

The enamel 204 and dentin 206 interface transmits forces exerted during chewing, while the periodontal ligament space 216 allows for the dissipation of these forces, preventing damage to the underlying alveolar bone 222 and by extension, the jawbone. The integration of these structures facilitates oral health and clear imaging of these components is important for surveying, analyzing and diagnosing dental and oral health.

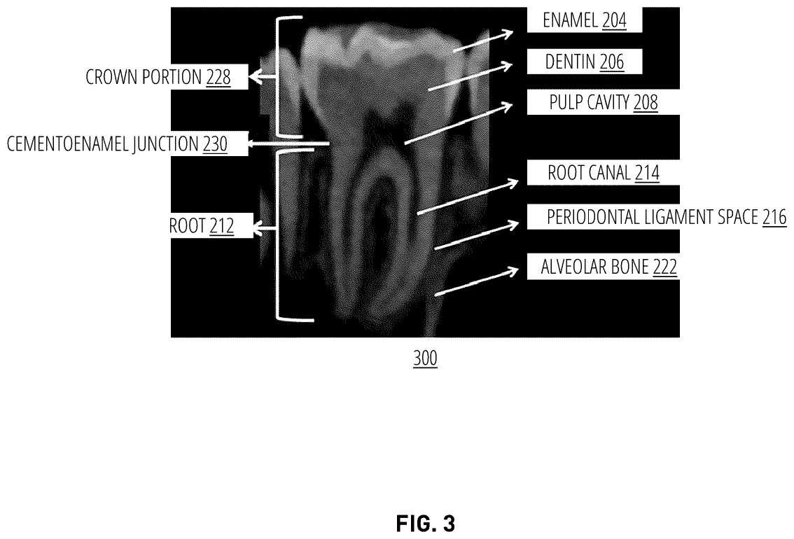

In FIG. 3, there is shown a radiographic image 300 of a tooth 202 similar to that illustrated in FIG. 2. The substructures of the enamel 204, the dentin 206, the cementum 226, the pulp cavity 208 or chamber and the periodontal ligament space 216 are all visually distinct from one another as are other structures such as the root canal 214 and the alveolar bone 222.

The radiographic image shown in FIG. 3, and other radiographic images, provides an important diagnostic tool for clinicians as it allows for visualization of the internal structure of tooth 202 and surrounding tissues. The image aids in identification of the border between the imaged anatomical structures, facilitating diagnosis and treatment planning.

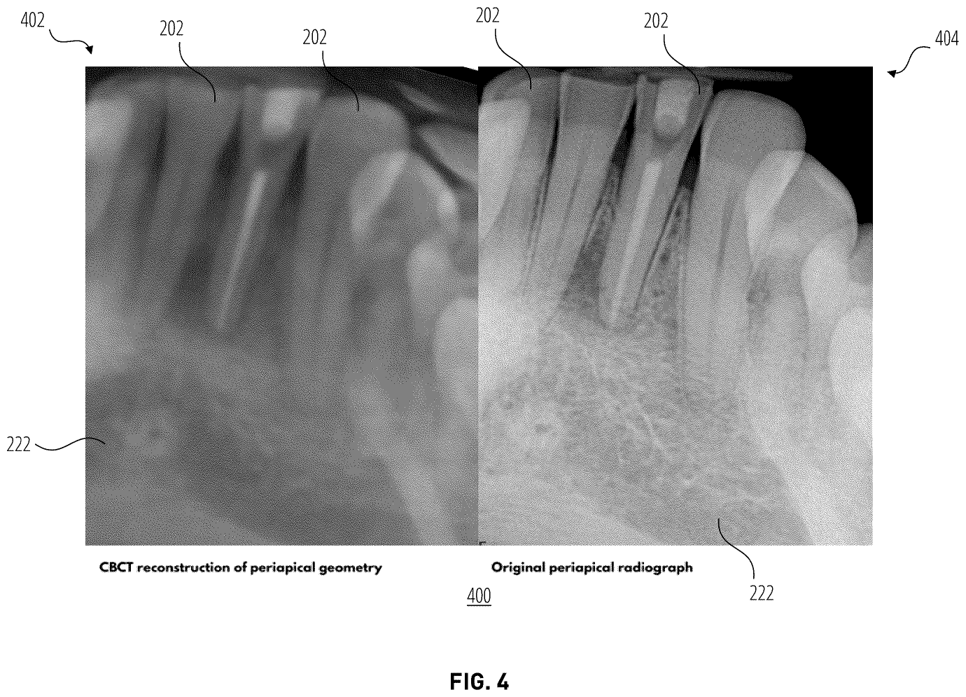

FIG. 4 is a juxtaposition 400 of a thickened Cone Beam Computed Tomography (CBCT) scan image 402 with a plain film periapical radiograph image 404 having the same view geometry. Juxtaposition 400 clearly shows that the CBCT scan reconstruction 402 is much blurrier than the plain film image 404. Each image shows a number of teeth 202 with associated substructures embedded in alveolar bone 222.

CBCT scan images 402 are generated typically using a cone-shaped X-ray beam that rotates around the patient, capturing multiple images from different angles. This series of images are then used to reconstruct a three-dimensional representation of the target area. This technology allows for detailed visualization of bone structure, dental anatomy, and other specific regions of interest.

While the resolution and quality of CBCT scan images is high, typically providing sub-millimeter spatial resolution, they are subject to limitations that can affect their diagnostic utility. These shortcomings are primarily related to the inherent nature of the imaging technology, patient-related factors, and technical limitations.

CBCT scans can suffer from scatter radiation, which occurs when the X-rays bounce off dense structures before reaching the detector, causing a decrease in image contrast and clarity. Beam hardening, a phenomenon that occurs when different tissues absorb X-rays at varying degrees, can lead to artifacts and distortion in the images. These effects are particularly noticeable around metal objects, such as dental fillings, implants, or appliances such as braces which can create streaks or shadows that obscure details.

Although CBCT scanning offers sub-millimeter resolution, the voxel size (the smallest distinguishable cube-shaped part of the scanned volume) limits the fineness of detail that can be observed. Smaller voxels provide higher resolution but increase radiation dose and scanning time. Accordingly, there is a trade-off between achieving high-resolution images and keeping the radiation exposure to a minimum. In practice, this means that very fine anatomical details may not be as clearly visible in CBCT images compared to conventional medical CT scans, which can achieve higher resolutions but at a significantly higher radiation dose.

CBCT images can exhibit a certain degree of noise, particularly in lower dose settings. Noise refers to random variations of brightness or color information in images that can obscure the clarity of the anatomical structures being examined. It can be influenced by the quality of the detector, the scanning parameters, and the reconstruction algorithms used.

CBCT is particularly useful for visualizing bony structures and teeth due to its high contrast resolution for hard tissues. However, its ability to differentiate between types of soft tissues is limited. This limitation can affect the diagnostic capabilities for soft tissue pathologies or the detailed assessment of structures with similar densities.

As with any imaging modality that requires a certain amount of time to acquire images, patient movement during the scanning process can also lead to blurring and artifacts, reducing the overall quality and diagnostic value of the images. Even minor movements can significantly impact the clarity of the resulting scans.

Plain film periapical radiograph images 404 are generated using traditional X-ray technology, where a focused beam of X-rays is passed through the area of interest and captured on a film or digital sensor placed on the opposite side. This produces a two-dimensional image that represents the density and composition of the structures within the path of the beam. The technique is particularly useful for examining individual teeth and surrounding bone in dental diagnostics.

The resolution of plain film periapical radiographs is generally high, allowing for detailed observation of the tooth structure, including the crown, root, and surrounding bone. However, these images only provide a two-dimensional view, which can limit the ability to assess complex anatomical relationships or pathologies that require three-dimensional understanding.

With reference to FIG. 4, it can be seen that existing CBCT scans are not ideal for creating images for simulation of 2D images of intraoral or extraoral plain films. The resolution of CBCT scans is too low to produce images of adequate quality for this purpose.

The high resolution and clarity of plain film periapical radiograph images or radiographic image 300 of FIG. 3, makes them more useful as training data for training a machine learning model which classifies regions of the image and labels them as substructures of a tooth 202 and/or surrounding tissue, which trained model or another trained model might also be used to diagnose conditions or provide treatment plans since structures and boundaries between structures are clearly identifiable. However, plain film periapical radiographs lack the three-dimensional quality of CBCT scans. Accordingly, the comprehensive three-dimensional understanding of patient anatomy provided by CBCT scans that is so valuable for diagnostic imaging is not available. Moreover, such training data is limited by the availability of clear images and the images which are available might not cover an extensive range of conditions, particularly in the case of rare conditions or abnormalities.

While the availability of clear radiographic images such as the radiographic image 300 of FIG. 3 or the periapical radiograph 404 of FIG. 4 may be limited in quantity, such images can serve as the basis for construction of a three-dimensional (3D) model from which can be retrieved any number of synthetic images with clear delineation between structures and which are appropriately labeled or annotated for the purposes of training a machine learning model. The 3D model can be constructed from a small subset of images and as few as a single image. This in turn, allows for training of the machine learning model to identify abnormalities such as dental caries, periodontal disease, and other pathologies like cysts, tumors, and bone abnormalities and their respective stage of development or for analysis of tooth positioning, bone structure, and growth patterns, all of which facilitate the crafting of accurate diagnoses and effective treatment strategies. Moreover, since any number of images can be retrieved from the 3D model, there is provided access to a wider array of training data for rare conditions. Thereby, the quantity of image information of a CBCT scan can be visualized in such a way that the clarity and quality of plain film scan view geometry is recreated, despite the resolution issues presented by the regular CBCT scan as shown in FIG. 4.

According to one aspect, there is provided a simulated, high-resolution three-dimensional (3D) model 500 from which may be acquired views of patient anatomy from various perspectives and in any number, as would be enabled from CBCT scan 402 or plain film scan with the same or better quality as compared to a 404.

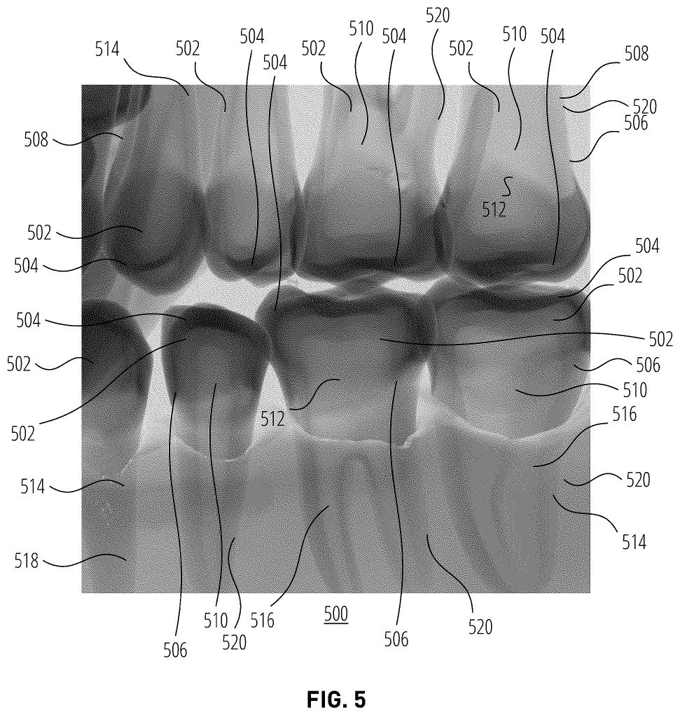

As shown in FIG. 5, the 3D model 500 is a synthetic 3D model 500 of varying opacity that mimics the transmissive properties of human tissue as it would appear in x-ray images, such as that shown in plain film periapical radiograph image 404 of FIG. 4 and radiographic image 300 of FIG. 3. From the 3D model 500, there can be produced any number of cross-sectional images or “slices” which are suitable for use as training data for training a machine learning model or deep learning system for the identification and segmentation of various conditions in a real x-ray image of a real patient's anatomy. Each slice is of similar or better clarity than plain film periapical radiograph image 404 of FIG. 4.

The 3D model 500 can be produced in a number of ways. In one aspect, the radiographically distinct substructures of one or more teeth 202 and surrounding structures are independently modeled to produce modeled teeth 502. The modeled substructures include the modeled enamel 504, the modeled dentin 506, the modeled cementum 508, the modeled pulp cavity 510 with modeled pulp therein, modeled root canal 516, modeled alveolar bone 518 and the modeled periodontal ligament space 520. Any other substructure of the teeth 202 or surrounding structures may also be modeled. Every radiographically distinct material is preferably independently modeled in 3D in order to produce the synthetic 3D model 500. In addition, all non-connected structures of the same radiodensity are preferably independently modeled. Furthermore, each individual structure or substructure is preferably organized and tracked independently in order to facilitate subsequent labeling and/or annotation.

In another aspect, the 3D model 500 is produced using patient data as a baseline. In this aspect, image data of patient scans is sufficiently accurate for generating a synthetic 3D model 500 directly therefrom. This may be done, for example, by compiling image data taken from a series of scan images of a patient from various angles. The images may then be analyzed by a human operator, by software, or both to identify features. The depth and spatial relationships between features are then identified. Appropriate stitching or reconstruction procedures may be used to assemble a volumetric 3D model of the patient data. In another aspect, different viewpoints may be used to construct a point cloud which represents the shape of the patient's anatomy. These points are then connected to form a mesh, which is a collection of vertices, edges and faces, that outline the geometry of the patient's anatomy in three dimensions. Finally, textures of the images taken from the patient may be mapped onto the 3D model 500, providing a realistic appearance and volume. A two-dimensional analogy for the process would be the tracing of a bitmap image with splines to produce a vectorized final output. The vector tracing has curves and features that would be infinitely scalable, whereas the bitmap source would have a degraded appearance at high zooms. The “vectorization” of the voxelized (bitmap) patient information contained in CBCT scans by the creation of 3D meshes with continuous surfaces that can be magnified indefinitely without loss of fidelity. In addition, this “vectorization” of voxel data permits nuanced editing of 3D contours to produce a perfect visual result, similar to how vectorization of bitmapped data permits the vector to be edited to produce a precise result.

In another aspect, patient image data may be used as a rough guideline for constructing the 3D model 500 and the specific substructures may be procedurally generated. Accordingly, patient-specific image data may be “generalized” to serve as a 3D representation of the dentoalveolar structures common to most people. Procedural generation uses computing to algorithmically generate the data used to define certain substructures. This is especially useful where acquired data is inaccurate or unclear. Procedural generation defines the parameters of the data to be generated and then the data defining those substructures is generated within those parameters. This is useful for modeling specific areas, such as the periodontal ligament space 216 around the teeth 202, the enamel 204 and the pulp 210 where patient data is often insufficiently accurate to produce a clear 3D model 500. In such instances, the 3D model 500 of the tooth 202 may be utilized as a baseline for procedurally generating a uniformly visible periodontal ligament space between the portion of the 3D model 500 defining a tooth 202 and the portion of the 3D model 500 defining the alveolar bone 222. Procedural generation may be used to generate other substructures as well. An example of a location where the patient data is typically adequate for 3D modeling is the trabecular and cortical structure of the mandibular and maxillary bone. In these cases, the bone structure may be simply defined inside the imaging data taken from the patient by a user-defined threshold and then converted to a 3D model 500, as the rough estimation of trabecular structure is adequate in such instances, and the procedural generation of a similar structure may produce an inferior result.

Whether constructed directly, using patient data as a baseline, or by procedural generation, the 3D model 500 is preferably composed of volumetric units such as voxels or polygonal meshes which provide for realistic representation of the internal substructures of the 3D model 500.

In FIG. 5, there is shown a 3D model 500 according to aspects described herein. As can be seen, the 3D model 500 is a synthetic model having a black and white gradient. An actual x-ray scan image is composed of a black and white gradient. The black and white gradient of the 3D model 500 allows for training images to be produced which are similar in appearance to actual x-ray scan images. Therefore, the black and white gradient image resulting from the transmissive properties of the x-rays used to generate the patient anatomy scans may be represented in the synthetic 3D model that relies on calculations of light transmission through solid 3D meshes of varying transmissivity.

The synthetic 3D model 500 includes a plurality of modeled teeth 502 and modeled alveolar bone 518 having each of the modeled teeth 502 embedded therein. Each modeled tooth 502 includes clearly defined modeled substructures, including modeled enamel 504, modeled dentin 506, modeled pulp cavity 510, modeled periodontal ligament space 520, and modeled root 514 with modeled root canal 516.

In real radiological scans of the patient, structures which are denser, either by nature of their thickness or the density of the material being scanned, attenuate the x-rays passing therethrough to a greater degree, resulting in a more intense appearance of the pixels or voxels in the resulting image. In a typical x-ray scan image this greater intensity may be shown by pixels or voxels which are darker or brighter, depending on the processing of the image data. One advantageous aspect of the synthetic 3D model 500 is that the synthetic 3D model reflects the higher attenuation information of structures which would have greater attenuation in real radiological scans of the patient. For example, the modeled enamel 504 of the modeled teeth 502 is modeled using mesh or voxels which is/are darker in the synthetic 3D model 500 shown in FIG. 5. This is because the enamel 204 of the teeth 202 scanned to create the 3D model 500 has higher density than other structures of the scanned teeth 202 and would appear more intensely than other less dense materials in a radiographic image.

Also, in some perspectives such as the perspective shown in FIG. 5, the modeled enamel 504 is viewed in cross-section. The x-rays used to produce this cross-section perspective are directed toward the patient's teeth generally in parallel with the teeth and passed through the enamel of the patient's teeth generally in parallel with the surfaces of the scanned teeth. Accordingly, those x-rays passed through the enamel 204 of each tooth 202 in its thickest direction. This results in the modeled enamel 504 having voxels with a very dark appearance in the synthetic 3D model 500. Likewise, the modeled substructures of the modeled teeth 502 having lower density, such as the modeled pulp cavity 510 and modeled dentin 506 have a lighter appearance in the synthetic 3D model than the modeled enamel 504 because the corresponding structures of the teeth scanned to create the 3D model 500 have a lower density than the enamel of the scanned teeth and hence attenuate x-rays passing therethrough to a lesser degree.

Since x-ray images are produced based on attenuation of x-rays, denser materials or thicker materials appear darker in original, non-inverted images than less dense or thin materials. This is because materials which are denser or thicker will attenuate x-rays more strongly than materials which are less dense or thinner. However, x-ray images which are viewed by a radiologist may be color-inverted or contrast-inverted so that the image appears similar to the way a natural bone or tooth would appear. That is, the enamel 204 appears brighter or whiter and structures having areas of lower density, such as the root 212 of a tooth 202, appearing darker or blacker.

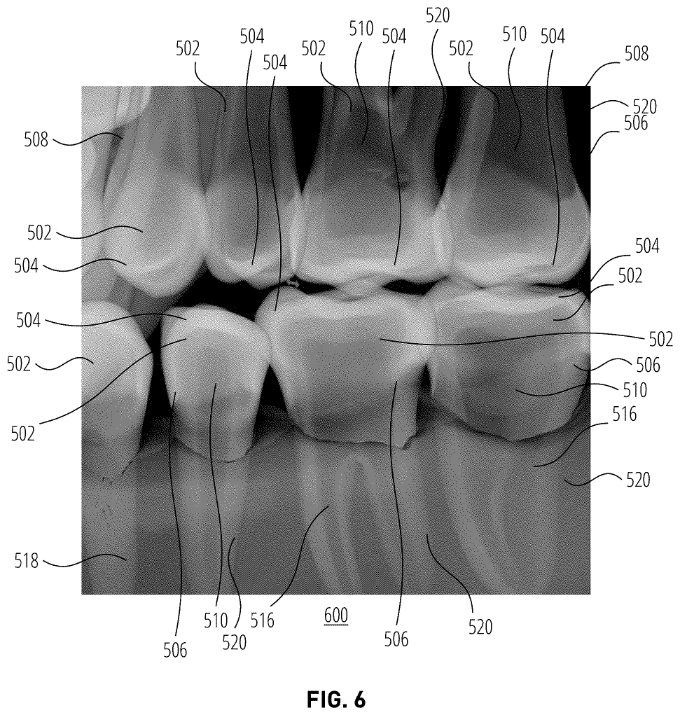

FIG. 6 shows inversion 600, wherein the coloration or contrast of the synthetic 3D model 500 is inverted in order to produce the inverted black and white or greyscale gradient as would appear in radiological images of patient dentoalveolar structures. Accordingly, there is provided a synthetic 3D model 500 based on patient scan data which reflects the black and white gradient image generated by x-ray imagery. This is advantageous as images extracted from a synthetic 3D model 500 colorized in this manner may be useful for a machine learning model being trained to interpret images of a same or similar color gradient.

The 3D model 500 has consistent clear resolution throughout the volume of each substructure. Accordingly, substructures which are obscured by layers of other substructures are not blurred or subject to other distortions which affect real CBCT scans. If it is desired, substructures may be rendered semitransparent or fully transparent in order to render the underlying substructures more apparent.

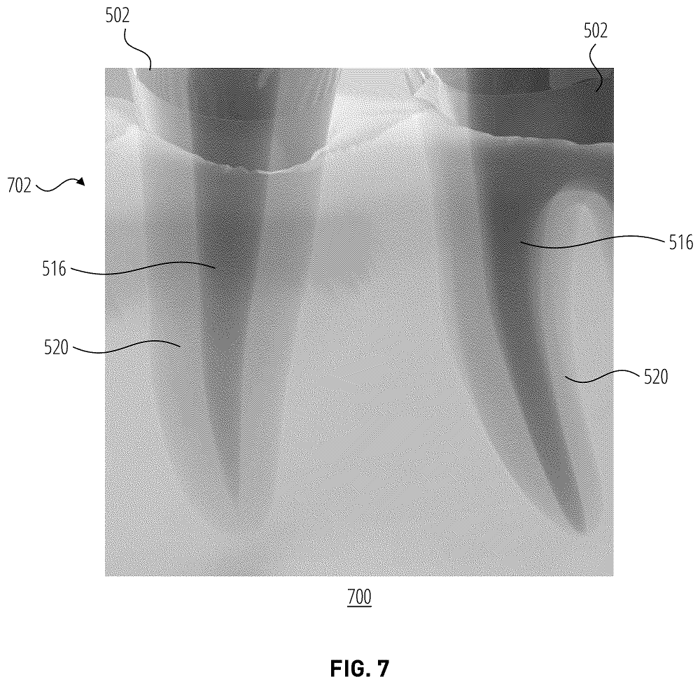

In FIG. 7, there is shown a magnified portion 700 of a 3D model 500. Model portion 700 shows a modeled dentoalveolar structure 702 including modeled teeth 502 embedded in a transparent modeled alveolar bone 518 without internal trabecular structure. Each tooth 202 includes modeled root 514 with modeled root canal 516 passing therethrough, modeled periodontal ligament space 520 extending between the modeled root and the modeled alveolar bone 518. Although not shown in the portion 700, the modeled teeth 502 may include other modeled substructures such as modeled enamel 504, modeled dentin 506, modeled pulp 512 and modeled periodontal ligament space 520. Shown in greyscale, the 3D model 500 is similar to a transmissive x-ray scan image.

The modeled alveolar bone 518 is at least partially transparent, as is the modeled root 514. Accordingly, both structures are highly visible, but the modeled root canal 516 is also very apparent because it is dark. This may be done by darkening individual substructures, where desired. Identification of substructures to be darkened, such as the modeled root canal 516, may be done by selecting data from the model annotated as being part of the modeled root canal 516 and then applying darker coloration to that data. Accordingly, although no layers of the 3D model 500 have been removed, all structures are clearly visible with high resolution.

Color may be applied to the 3D model 500 and/or substructures thereof in order to make features of the 3D model 500 more apparent. The data making up the 3D model 500 is annotated. Color may be applied to data of a selected type, that is data bearing certain annotations selectable by a user, while other data is left unaffected or may be differently colored. Not only can the color or contrast of substructures be modified, but also the transparency. Thereby, substructures of the 3D model 500 may be made more or less visible to a human person looking at images taken from the 3D model 500 or to a machine learning algorithm being trained using the 3D model 500 or on images taken therefrom.

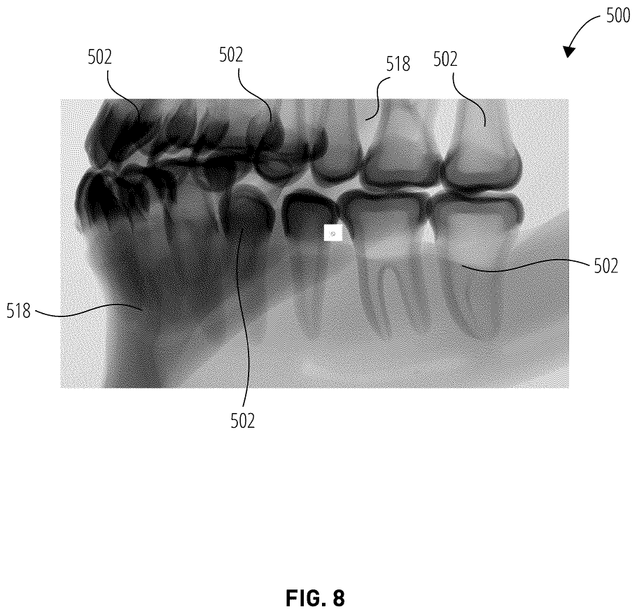

In FIG. 8, there is shown a 3D model 500 having modeled teeth 502 and modeled alveolar bone 518 according to one aspect having shading and transparency which more realistically depicts the transmissive shading and attenuation of light by the materials that comprise the various calcified structures of the teeth 202 and jaws.

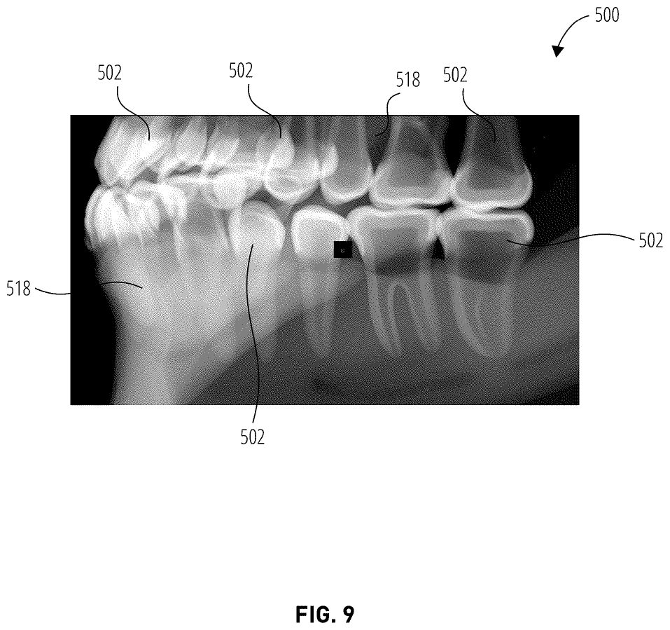

FIG. 9 shows the image coloration of FIG. 8 inverted to provide the typical appearance of a dental x-ray image. Accordingly, images or “slices” taken from the 3D model 500 will have the appearance of a dental x-ray image. Any number of slices may be taken from any desired angle or orientation in order to provide a robust body of training data for a machine learning model being trained to view and interpret x-ray images.

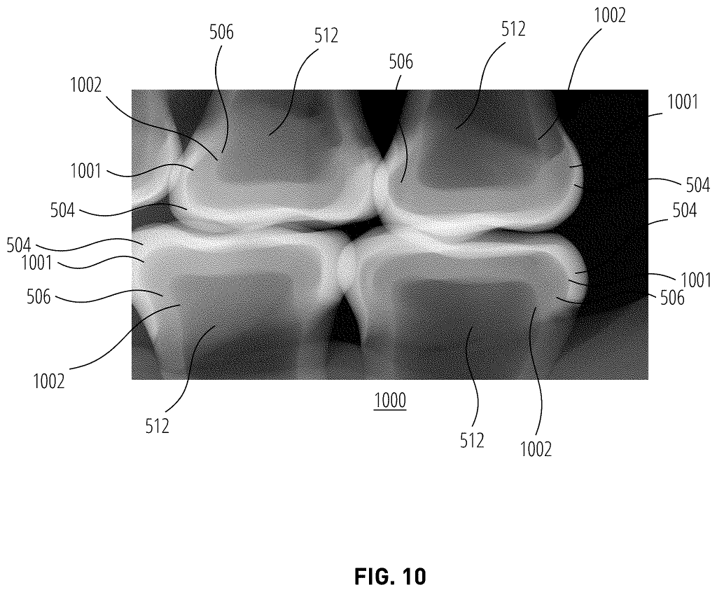

FIG. 10 is a portion 1000 of FIG. 9 which has been magnified in order to illustrate the smooth contours and high-resolution images available using the synthetic 3D model 500, according to one aspect. Such high-resolution images provide smooth contours which cannot be achieved using CBCT scans 402 as source data. The delineations between the modeled teeth 502 and their respective modeled substructures are clear and distinct in the synthetic 3D model 500 and may be as distinct as the resolution of the image allows.

For example, a first delineation 1001 between the modeled enamel 504 and the modeled dentin 506 is easily identified. Moreover, a second delineation 1002 between the modeled dentin 506 and the modeled pulp 512 is also easily identified. These clear delineations exist between all structures of the modeled teeth 502 shown in the synthetic 3D model 500. Moreover, the clear delineations are maintained for different levels of magnification or zoom and for all modeled teeth 502 from any perspective in the synthetic 3D model 500. This level of sharpness is advantageous for training machine learning models because the structures of the modeled teeth 502 may be labeled with greater consistency and accuracy.

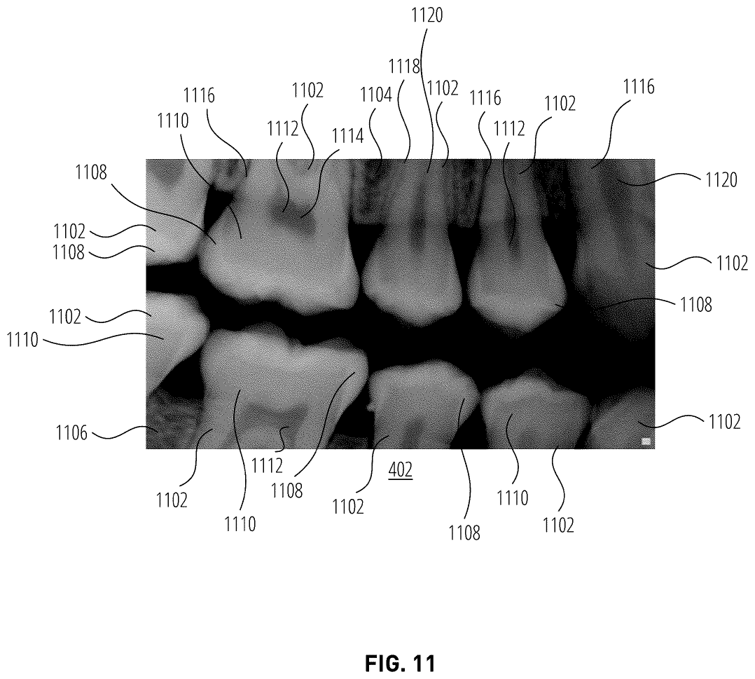

By way of contrast, FIG. 11 illustrates a real CBCT scan image 402 in the form of a bitewing radiograph which has contours which are “fuzzy” and less sharp or precise as those provided using the synthetic 3D model 500. A machine learning model trained using CBCT scan images 402, such as that shown in FIG. 11, would have less accurate output since the delineations between structures of the teeth are more difficult to identify.

Shown in FIG. 11 is a plurality of imaged teeth 1102 embedded in an imaged upper jaw bone 1104 and an imaged lower jaw bone 1106. As with previous dental images described herein, structures of imaged teeth 1102 include imaged enamel 1108, imaged dentin 1110, imaged pulp cavity 1112 containing imaged pulp 1114, imaged alveolar ligament space 1116, imaged root 1118 and imaged root canal 1120 passing therethrough.

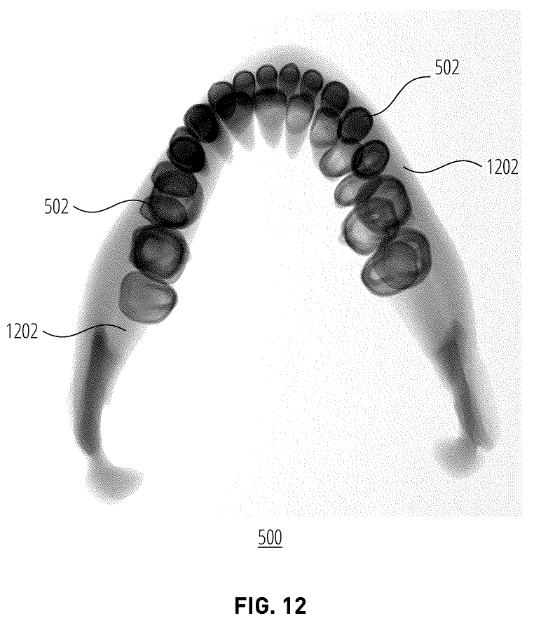

In FIG. 12, there is shown a perspective view of a synthetic 3D model 500, according to one aspect, which includes modeled upper jaw bone 1202 and modeled lower jaw bone 1204 with modeled teeth 502 embedded therein, viewed from the top down. As explained above, the real transmission attenuation properties of x-ray images can be reflected in the synthetic 3D model 500. This is particularly true where the 3D model 500 is generated from real x-ray image data. This provides advantage wherein perspective views of the synthetic 3D model 500 more realistically reflect attenuation of x-rays as it would appear in x-ray imaging. For example, the modeled teeth 502 shown in FIG. 12 appear much more opaque when stacked on their long axes due to the thickness of the teeth along this axis. A real transmission x-ray image of teeth along the same axis would result in images of the teeth having higher opacity.

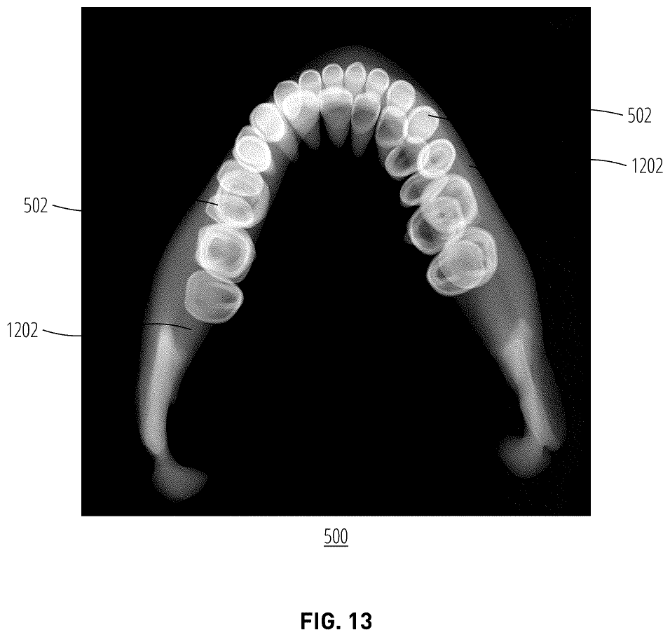

FIG. 13 is the synthetic 3D model shown in FIG. 12 with inverted coloration. The synthetic 3D model 500 provides images having smoother shading than conventional non-synthetic images, realistic representation of attenuation properties of individual objects, an organic appearance of the contours, and an overall high-definition composition resulting in a high-quality, realistic mimic of a real x-ray of the mandible. The synthetic 3D model may be used to capture any perspective view or cross-section for the purpose of generating new images suitable for use in training of machine learning models or deep learning models for diagnostics and dental diagnostics, in particular.

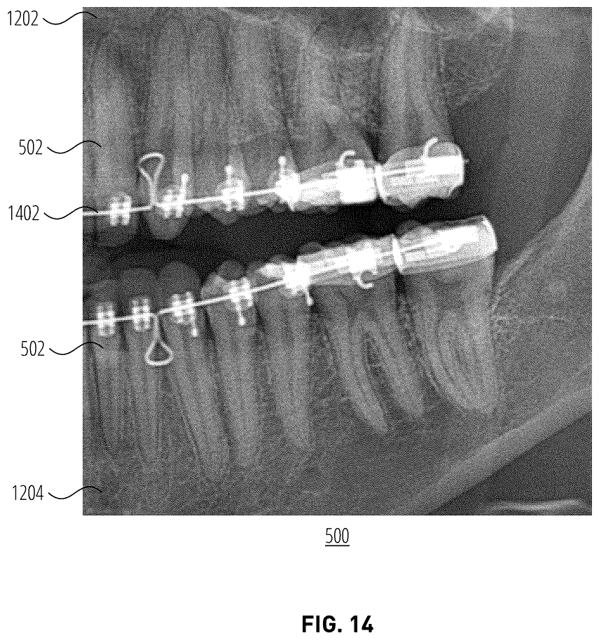

In FIG. 14, there is shown another aspect wherein the 3D model 500 includes at least one modeled abnormality 1402. Modeled abnormality 1402 may include any dental pathology, caries, teeth or other structures in various stages of development or the presence of dental appliances such as retainers, braces or other devices. In the aspect shown in FIG. 14, modeled abnormality 1402 is braces.

In the aspect of FIG. 14, there is shown modeled teeth 502 with modeled substructures thereof and modeled upper jawbone 1202 and modeled lower jawbone 1204 with emphasis on realistic representations of transmissive shading and attenuation of light by the materials that comprise the various calcified structures of the teeth 202 and jaws. The modeled upper jawbone 1202 and modeled lower jawbone 1204 have a lattice representative of the lattice of bone within the upper and lower jawbones, as it would appear in a high-resolution x-ray scan image.

The braces, shown by way of modeled abnormality 1402, are made of metal and have a very high attenuation. Accordingly, the braces are modeled as bright white in the 3D model 500 shown in FIG. 14 to reflect the intensity of their appearance in a radiographic image.

Also exemplified in the aspect shown in FIG. 14 is the high degree of resolution achievable by the 3D model 500. Modeling of the bone lattice or trabeculae of the interior of the jawbones requires a very high degree of resolution because such structures are very fine.

The 3D model 500 provides a rich source of images which may be used as training data for training machine learning models to view and interpret real radiographic images which may contain abnormalities such as dental appliances or very fine structures such as bone lattice. Such a rich source of training data would be time consuming and expensive to acquire in the absence of the 3D model 500.

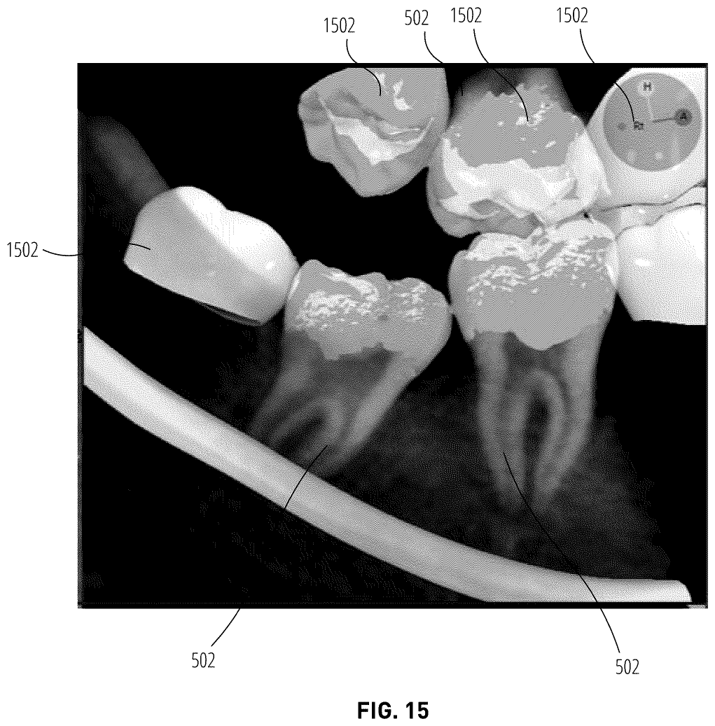

In the aspect of FIG. 15, there is shown a 3D model 500 with modeled abnormalities 1502 this time representing modeled crowns partially or fully encircling modeled teeth 502. The modeled crowns are shown with realistic shading and are also shown in contrast to the underlying modeled teeth 502, thereby making the modeled crowns highly visible relative to the surrounding structures. This is useful not only for a practitioner working with the synthetic 3D model 500 but is also useful for training a machine learning model using the 3D model 500 because the data of the 3D model 500 is appropriately labeled and the structures representing the modeled crowns are easily identifiable to a machine learning model.

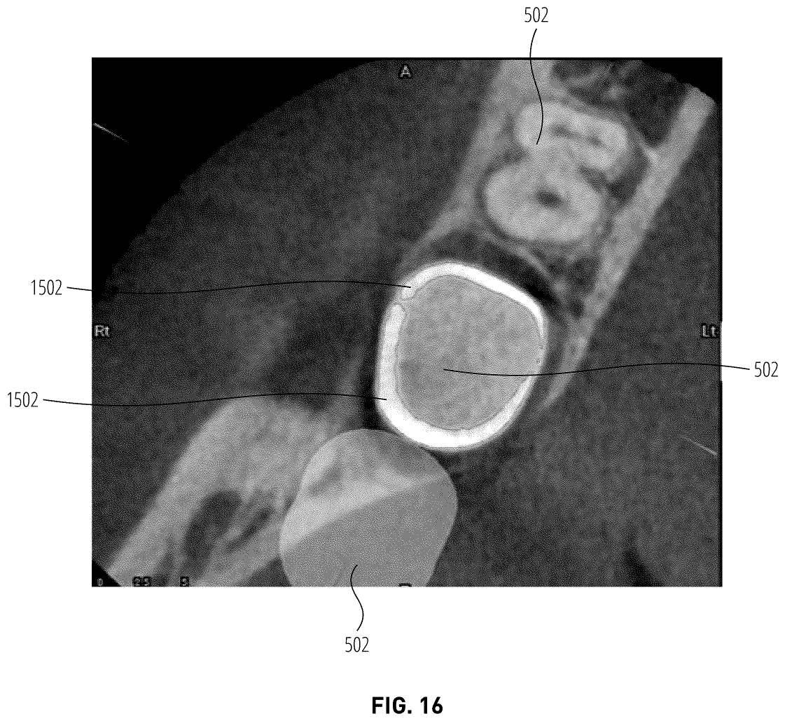

FIG. 16 is a magnified section of the 3D model 500 shown in FIG. 15 and with greyscale shading. The section view of FIG. 16 is taken along a plane parallel to an upper surface of the modeled teeth 502. Although magnified, the delineations between substructures of the 3D model 500 are well-defined and visible both to the naked eye and to a computer for image processing. Modeled abnormality 1502, the crown, is shown surrounding a modeled tooth 502. The delineation between the structure of the crown and the structure of the modeled tooth 502 is also well-defined. The annotation of data within the 3D model 500 permits for such clarity between substructures which would be very difficult if not impossible to clearly and accurately distinguish using radiographic images of the same substructures.

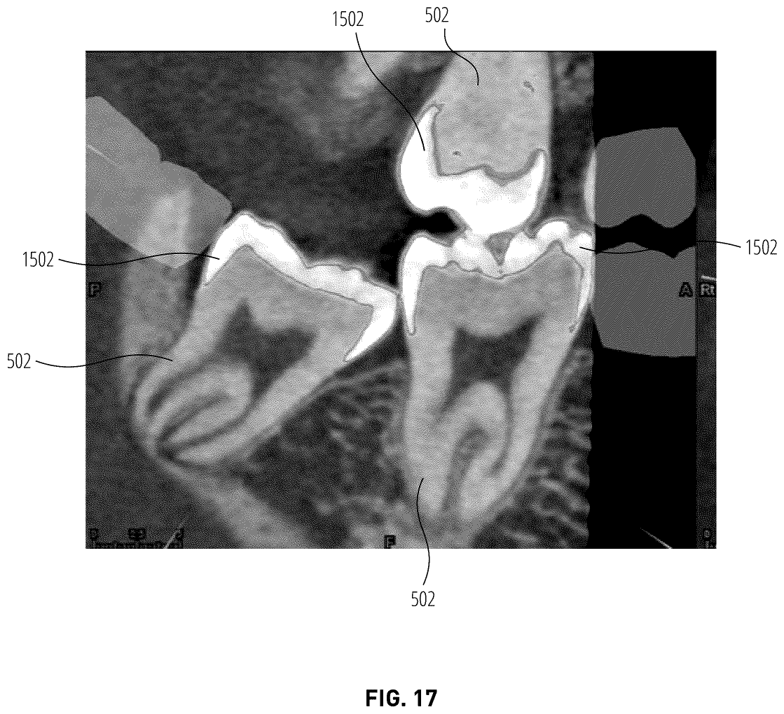

FIG. 17 is another magnified section view of the 3D model 500 shown in FIG. 15 and with greyscale shading. The section view of FIG. 17 is taken along a plane transverse to an upper surface of the modeled teeth 502. As with FIG. 16, the delineation between modeled teeth 502 and modeled abnormalities 1502 is very clear, even at high levels of magnification.

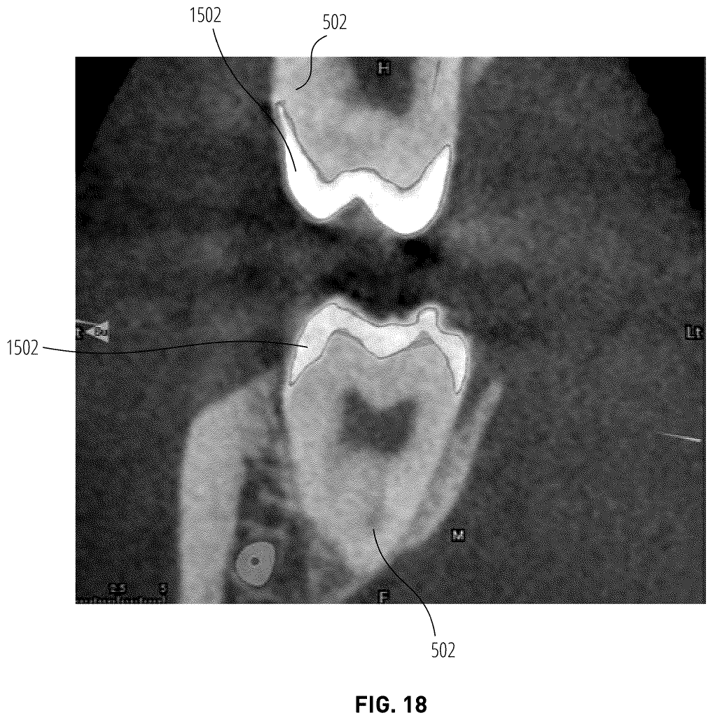

FIG. 18 is a section view of a 3D model 500 similar to that shown in FIG. 17, but at a higher level of magnification. Even at very high levels of magnification, the delineation between modeled teeth 502 and modeled abnormalities 1502 is very clear.

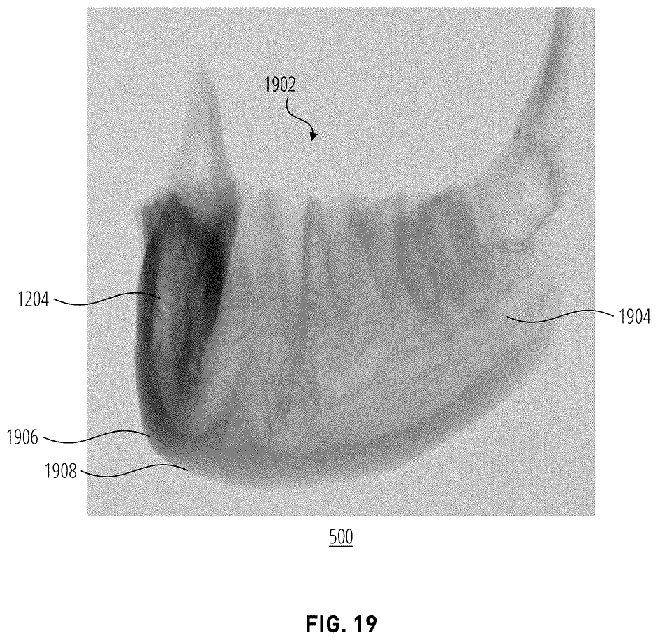

FIG. 19 illustrates a modeled lower jawbone 1204 with modeled teeth 502 removed, leaving modeled empty teeth sockets 1902. Visible in the modeled lower jawbone 1204 are the modeled trabeculae 1904, modeled lamina dura 1906 and modeled cortical boundaries 1908 of the modeled lower jawbone 1204.

The intensity of the contrast is proportional to the thickness of the cortical bone in the view direction, and therefore the 3D model 500 faithfully reproduces the appearance of the structure as it would appear in a transmission x-ray image of a mandible of a patient.

Accordingly, the 3D model 500 provides for modeling of very fine substructures of anatomical structures and also provides for selected structures to be removed, temporarily or permanently, from the 3D model 500. This allows for isolation of selected substructures and removes obstructions and layers that might otherwise interfere with some desired perspectives.

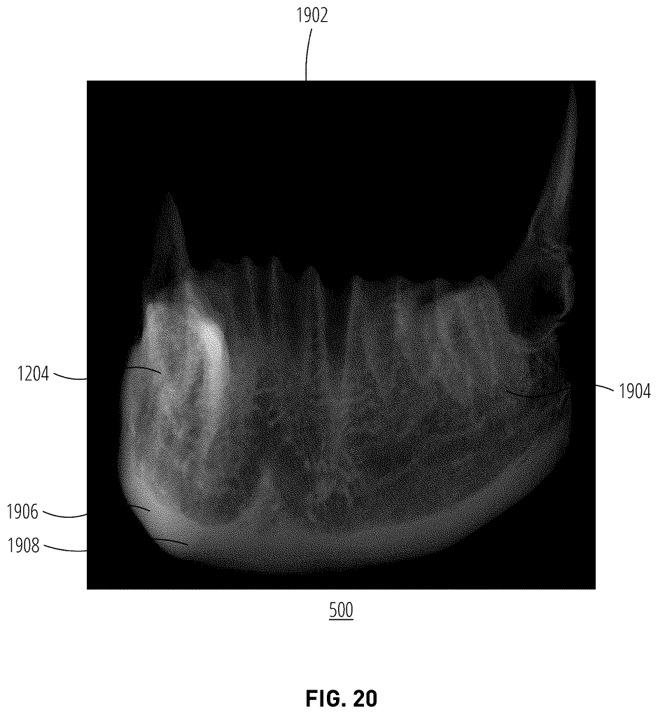

As with previous aspects, coloration or contrast of the aspect shown in FIG. 19 may be inverted. FIG. 20 is the synthetic 3D model 500 shown in FIG. 19 with inverted coloration.

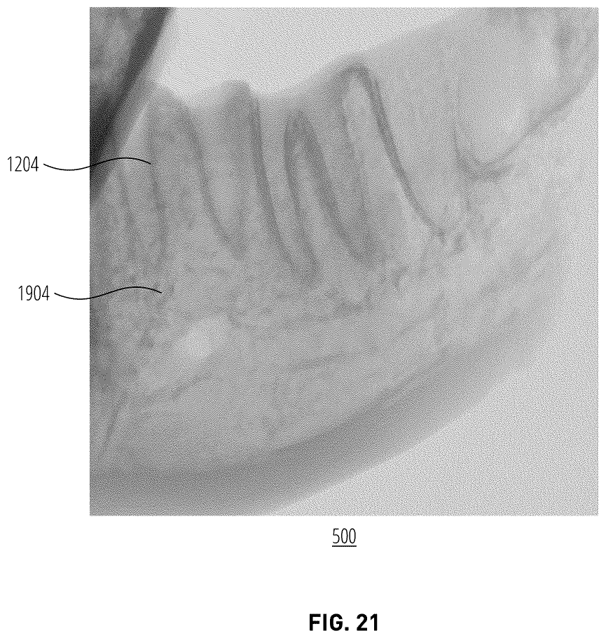

FIG. 21 is the synthetic 3D model 500 shown in FIG. 19 with greater magnification. Although some modeled trabeculae 1904 may appear mottled at higher magnification in some aspects, the boundaries of the modeled lower jawbone 1204 appear continuous and smooth.

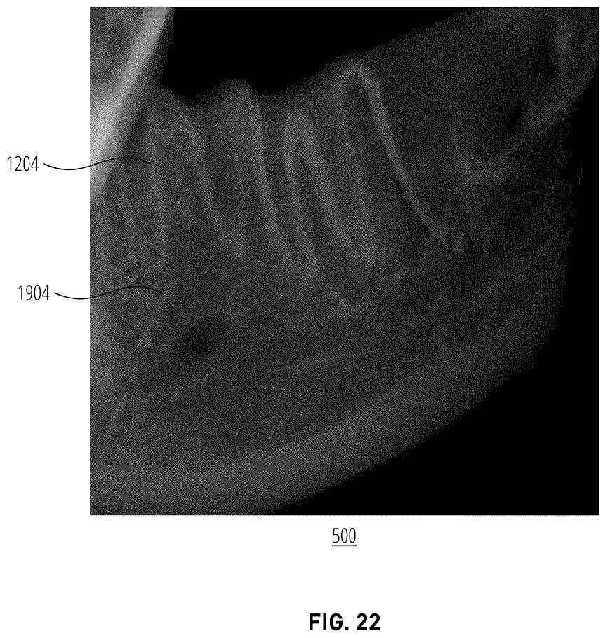

FIG. 22 is the image shown in FIG. 21 with inverted coloration.

One very useful feature of the synthetic 3D model 500 according to the aspects described herein is that the labeling or annotation of the data within the synthetic 3D model 500 may be done automatically with a high degree of accuracy. This can provide many ways in which visibility of modeled substructures or tissues within the 3D model 500 can be enhanced both for the benefit of a human view and for a machine learning model being trained using data from the 3D model 500.

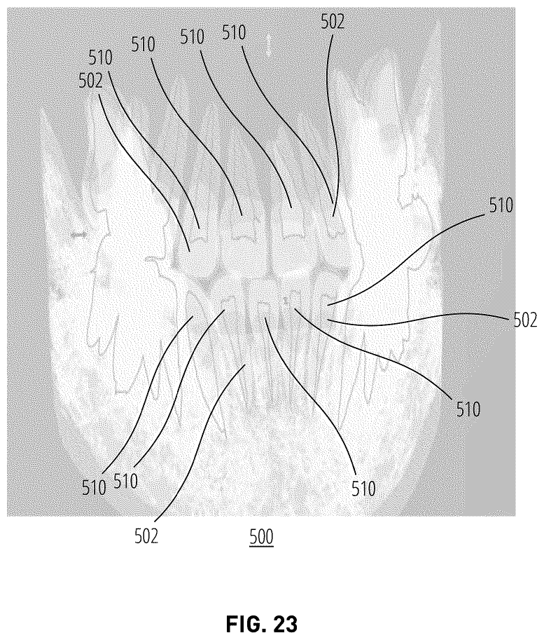

FIG. 23 illustrates one aspect wherein the modeled pulp cavities 510 of each modeled tooth 502 are outlined or traced with a high degree of accuracy. Defining the borders between structures in a radiographic image is a complicated and time consuming process wherein a human, typically with the assistance of software, must determine the boundaries separating two structures. In images with low resolution, many artifacts or other distortions, this can be challenging and often results in a “best guess” being made by the human or the software interpreting the image.

A 3D model 500 as described herein has accurate borders defined around each tissue and structure since the data is accurately labeled or annotated in building the model. Therefore, the 3D model 500 provides training data images with accurate borders between modeled structures and tissues so that the trained machine learning model may, in turn, define the boundaries between structures shown in radiographic images with a high degree of accuracy.

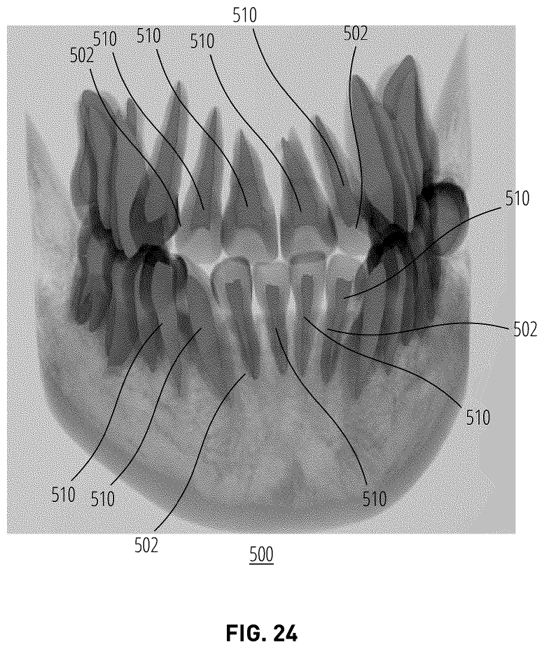

As shown in FIG. 24, the highlighting of the modeled pulp cavities 510 provides further nuance, based on whether items overlap with each other in the 3D model 500. FIG. 24 is a view of the 3D model 500 according to the aspect shown in FIG. 23, with further enhancement to highlight the modeled pulp cavity 510 of each modeled tooth 502. Several of the modeled pulp cavities 510 overlap with one another. In the image shown in FIG. 23, it may be difficult to visually determine the delineations of the modeled pulp cavities 510 between modeled teeth 502. With modeled pulp cavities 510 of each modeled tooth highlighted, those delineations and areas of overlap become much clearer. Accordingly, the data representing the modeled pulp cavity 510 of individual modeled tooth 502 may be more clearly identified to a machine. With clear labeling of the data, it becomes more efficient to train a machine learning model to identify which areas of an image indicate pulp, for example, and which do not. This principle applies to image data indicating other structures of the teeth and alveolar structures as well, since the delineations of these structures will also be clear and may also be highlighted to promote further efficiency during the training process.

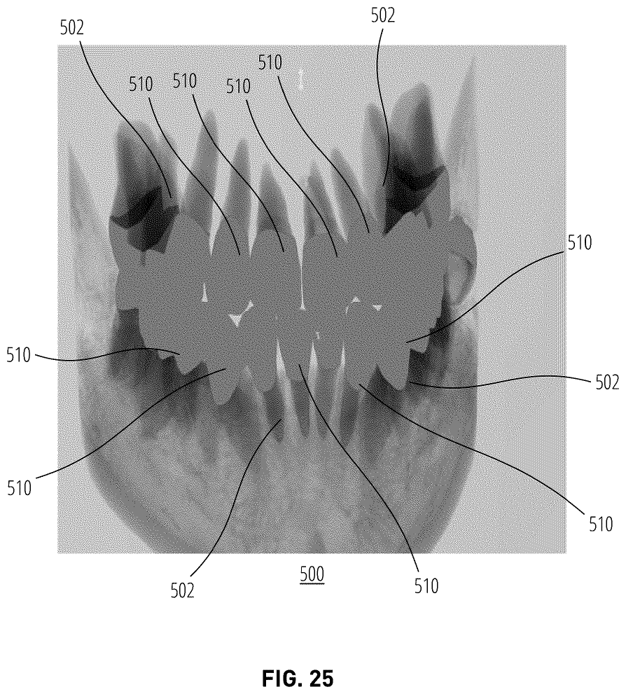

In FIG. 25, the 3D model 500 is shown with modeled enamel 504 highlighted, providing another perspective view wherein delineation between modeled teeth 502 and substructures thereof is visibly very clear.

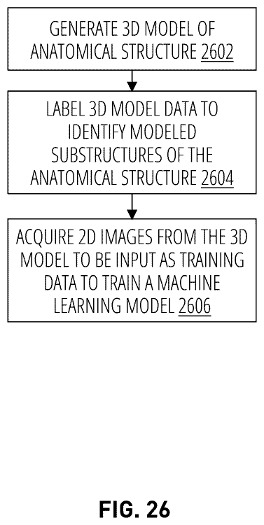

FIG. 26 illustrates a method for acquiring training data for training a machine learning model, according to one aspect. At block 2602, a three-dimensional model of at least one anatomical structure is generated. In one preferred aspect, the at least one anatomical structure is a dentoalveolar structure. However, the method may be applied to other anatomical structures. Also, the term “anatomical structure” typically refers to a bodily structure. However, as used herein, the term “anatomical structure” may also include appliances, implants or other artificial components that have been attached, adhered, grafted or otherwise attached to the anatomy of a person or animal from which the three-dimensional model is acquired. The three-dimensional model of the at least one anatomical structure may be generated or constructed according to any of the means described previously herein.

At block 2604, the data of the three-dimensional model is labeled to identify modeled substructures of the at least one anatomical structure. By way of non-limiting example, the substructures so labeled or annotated may include the modeled substructures previously described herein, such as the modeled enamel, modeled dentin, modeled pulp cavity, modeled pulp, modeled alveolar ligament space, modeled root cavity, modeled appliance, modeled pathology, modeled growth or any other modeled abnormality.

At block 2606, a plurality of two-dimensional images are acquired from the three-dimensional model. These two-dimensional images are for use in training a machine learning model useful for identification of substructures of the at least one anatomical structure in an image. The two-dimensional images may be acquired using any of the means previously described herein. For example, the two-dimensional image may be a section or cross-section of the three-dimensional model or other perspective image useful as training data for training a machine learning model.

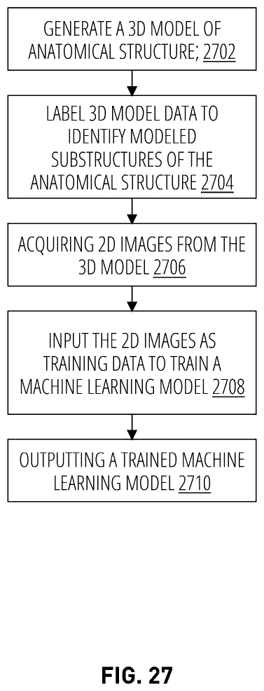

FIG. 27 illustrates a method for training a machine learning model, according to one aspect. At block 2702, a three-dimensional model of at least one anatomical structure is generated. In one preferred aspect, the at least one anatomical structure is a dentoalveolar structure. However, the method may be applied to other anatomical structures.

At block 2704, the data of the three-dimensional model is labeled to identify modeled substructures of the at least one anatomical structure. By way of non-limiting example, the substructures so labeled or annotated may include the modeled substructures previously described herein, such as the modeled enamel, modeled dentin, modeled pulp cavity, modeled pulp, modeled alveolar ligament space, modeled root cavity, modeled appliance, modeled pathology, modeled growth or any other modeled abnormality.

At block 2706, a plurality of two-dimensional images are acquired from the three-dimensional model. These two-dimensional images are for use in training a machine learning model useful for identification of substructures of the at least one anatomical structure in an image. The two-dimensional images may be acquired using any of the means previously described herein. For example, the two-dimensional images may be sections or cross-sections of the three-dimensional model or other perspective image useful as training data for training a machine learning model. As described above, any number of section images or perspective views may be taken from a three-dimensional model for the purpose of acquiring training data.

At block 2708, the plurality of two-dimensional images are input as training data to train a machine learning model to identify substructures of the at least one anatomical structure in an image.

At block 2710, there is output a machine learning model trained to identify substructures of the at least one anatomical structure in an image.

A system of one or more computers or computerized architectural components can be configured to perform particular operations or actions by virtue of having software, firmware, hardware, or a combination thereof installed on the system that in operation causes or cause the system to perform the actions. One or more computer programs recorded on one or more computer storage devices can be configured to perform particular operations or actions by virtue of including instructions that, when executed by data processing apparatus, cause the apparatus to perform the actions automatically and/or in real-time.

While the invention has been described in terms of specific embodiments, it is apparent that other forms could be adopted by one skilled in the art. For example, the methods described herein could be performed in a manner which differs from the embodiments described herein. The steps of each method could be performed using similar steps or steps producing the same result, but which are not necessarily equivalent to the steps described herein. Some steps may also be performed in different order to obtain the same result. Similarly, the apparatuses and systems described herein could differ in appearance and construction from the embodiments described herein, the functions of each component of the apparatus could be performed by components of different construction but capable of a similar though not necessarily equivalent function, and appropriate materials could be substituted for those noted. Accordingly, it should be understood that the invention is not limited to the specific embodiments described herein. It should also be understood that the phraseology and terminology employed above are for the purpose of disclosing the illustrated embodiments, and do not necessarily serve as limitations to the scope of the invention.

What is claimed is:

1. A method for acquiring training data for training a machine learning model, comprising the steps of:
generating a three-dimensional model of at least one anatomical structure;
labeling data of the three-dimensional model to identify modeled substructures of the at least one anatomical structure; and,
acquiring a plurality of two-dimensional images from the three-dimensional model to be input as training data to train a machine learning model useful for identification of substructures of the at least one anatomical structure in an image.

2. The method of claim 1, wherein the step of acquiring a plurality of two-dimensional images further comprises the step of:
taking a plurality of cross-section images from portions of the three-dimensional model.

3. The method of claim 1, wherein the image is a radiographic image.

4. The method of claim 3, wherein the radiographic image is a radiographic image of at least one dentoalveolar structure of a patient.

5. The method of claim 1, wherein the step of generating a three-dimensional model of at least one anatomical structure further comprises the steps of:
acquiring a volumetric scan of the at least one anatomical structure; and,
constructing a three-dimensional model of the at least one anatomical structure.

6. The method of claim 5, wherein the step of constructing the three-dimensional model further comprises the step of:
manually constructing at least a portion of the three-dimensional model.

7. The method of claim 5, wherein the step of constructing the three-dimensional model further comprises the step of:
procedurally generating at least a portion of the three-dimensional model.

8. The method of claim 5, wherein the step of constructing the three-dimensional model further comprises the step of:
independently modeling each modeled substructure of the three-dimensional model and then assembling the modeled substructures together to form the three-dimensional model.

9. The method of claim 5, wherein the step of acquiring the volumetric scan further comprises the step of:
scanning at least one anatomical structure of a patient to acquire the volumetric scan.

10. The method of claim 1, wherein, after the generating step, the method further comprises the step of:
applying a greyscale gradient to voxels of the three-dimensional model.

11. The method of claim 10, further comprising the step of:
inverting the greyscale gradient.

12. The method of claim 1, wherein, after the generating step, the method further comprises the step of:
applying at least one color to voxels of the modeled substructures.

13. The method of claim 12, wherein each of the modeled substructures is independently colored.

14. The method of claim 1, wherein, after the generating step, the method further comprises the step of:
selectively rendering at least partially transparent voxels of the three-dimensional model.

15. The method of claim 1, wherein the three-dimensional model includes at least one modeled dental appliance.

16. The method of claim 1, wherein the three-dimensional model includes at least one modeled pathology.

17. The method of claim 1, wherein the modeled substructures include at least one modeled tooth structure including at least one of modeled enamel, modeled dentin, modeled pulp cavity, modeled pulp, modeled alveolar ligament space, and modeled root cavity.

18. The method of claim 1, wherein the modeled substructures include at least one modeled bone structure including at least one of modeled trabeculae, modeled lamina dura and modeled cortical boundaries.

19. The method of claim 1, further comprising the step of:
outlining at least one modeled substructure of the three-dimensional model.

20. The method of claim 1, further comprising the step of:
highlighting at least one modeled substructure of the three-dimensional model.

21. The method of claim 1, wherein modeled substructures are at least one of removeable and isolatable from the three-dimensional model.

22. A method for training a machine learning model comprising the steps of:
generating a three-dimensional model of at least one anatomical structure;
labeling data of the three-dimensional model to identify modeled substructures of the at least one anatomical structure;
acquiring a plurality of two-dimensional images from the three-dimensional model; and,
inputting the plurality of two-dimensional images as training data to train a machine learning model to identify substructures of the at least one anatomical structure in an image.

23. The method of claim 22, further comprising the step of:
outputting a machine learning model trained to identify substructures of the at least one anatomical structure in an image.

24. The method of claim 22, wherein the step of acquiring a plurality of two-dimensional images further comprises the step of:
taking a plurality of cross-section images from portions of the three-dimensional model.

25. The method of claim 22, wherein the image is a radiographic image.

26. The method of claim 25, wherein the radiographic image is a radiographic image of at least one dentoalveolar structure of a patient.

27. The method of claim 22, wherein the step of generating a three-dimensional model of at least one anatomical structure further comprises the steps of:
acquiring a volumetric scan of the at least one anatomical structure; and,
constructing a three-dimensional model of the at least one anatomical structure.

28. The method of claim 27, wherein the step of constructing the three-dimensional model further comprises the step of:
manually constructing at least a portion of the three-dimensional model.

29. The method of claim 27, wherein the step of constructing the three-dimensional model further comprises the step of:
procedurally generating at least a portion of the three-dimensional model.

30. The method of claim 27, wherein the step of constructing the three-dimensional model further comprises the step of:
independently modeling each modeled substructure of the three-dimensional model and then assembling the modeled substructures together to form the three-dimensional model.

31. The method of claim 27, wherein the step of acquiring the volumetric scan further comprises the step of:
scanning at least one anatomical structure of a patient to acquire the volumetric scan.

32. The method of claim 22, wherein, after the generating step, the method further comprises the step of:
applying a greyscale gradient to voxels of the three-dimensional model.

33. The method of claim 32, further comprising the step of:
inverting the greyscale gradient.

34. The method of claim 22, wherein, after the generating step, the method further comprises the step of:
applying at least one color to voxels of the modeled substructures.

35. The method of claim 34, wherein each of the modeled substructures is independently colored.

36. The method of claim 22, wherein, after the generating step, the method further comprises the step of:
selectively rendering at least partially transparent voxels of the three-dimensional model.

37. The method of claim 22, wherein the three-dimensional model includes at least one modeled dental appliance.

38. The method of claim 22, wherein the three-dimensional model includes at least one modeled pathology.

39. The method of claim 22, wherein the modeled substructures include at least one modeled tooth structure including at least one of modeled enamel, modeled dentin, modeled pulp cavity, modeled pulp, modeled alveolar ligament space, and modeled root cavity.

40. The method of claim 22, wherein the modeled substructures include at least one modeled bone structure including at least one of modeled trabeculae, modeled lamina dura and modeled cortical boundaries.

41. The method of claim 22, further comprising the step of:
outlining at least one modeled substructure of the three-dimensional model.

42. The method of claim 22, further comprising the step of:
highlighting at least one modeled substructure of the three-dimensional model.

43. The method of claim 22, wherein modeled substructures are at least one of removeable and isolatable from the three-dimensional model.
